(19) United States
(12) Patent Application Publication
(10) Pub. No.: US 2005/0095239 A1
(43) Pub. Date: May 5, 2005

(54) Methods and compositions for treating Alzheimer's disease

(76) Inventors: G. William Rebeck, Washington, DC (US); Amy Deng, Charlestown, MA (US); Hiroaki Fukumoto, Charlestown, MA (US); Michael Irizarry, Charlestown, MA (US); Michael Fitzgerald, Boston, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/664,414

(22) Filed: Sep. 17, 2003

Related U.S. Application Data
(60) Provisional application No. 60/411,706, filed on Sep. 18, 2002.

(51) Int. Cl. A61K 39/395

(52) U.S. Cl. 424/143.1

(57) ABSTRACT

The present invention provides methods for reducing the efflux of cholesterol and/or secreted Aβ in the brain, as well as methods for treating Alzheimer's disease, by inhibit the expression and/or activity of LXR and/or ABCA1. Compositions comprising agents for performing the methods also are provided.

### RELATED APPLICATION

This application claims the benefit under 35 U.S.C. § 119 of U.S. provisional patent application 60/411,706, filed Sep. 18, 2002.

### GOVERNMENT SUPPORT

This work was funded in part by the National Institutes of Health under grant numbers R01 AG14473, K08 AG00793, HL68988 and HL10398. Accordingly, the government may have certain rights in this invention.

### FIELD OF THE INVENTION

The present invention relates to methods and compositions for modulation of cholesterol efflux in brain cells, for reducing secretion of Aβ in brain cells, and for treatment of Alzheimer's disease.

### BACKGROUND OF THE INVENTION

Alzheimer's disease (AD) is a late onset neurodegenerative disorder characterized by the extracellular deposition of insoluble aggregates composed of the 40 to 42 amino acid AP peptide in the brain (Glenner and Wong, Biochem. Biophys. Res. Commun. 120:885-890 (1984); Masters et al., EMBO J. 4:2757-2763 (1985)). Aβ peptide is derived from an integral membrane protein termed amyloid β-protein precursor protein (APP) (Tanzi et al., Science 235:880 (1987); Kang et al., Nature 325:733-736 (1987)). The function and metabolism of APP have been the subject of intensive study due to the fact that mutations in APP are associated with an autosomal dominant form of AD, (Goate et al., Nature 349:704-707 (1991)) and over-production of APP is the presumptive cause of AD in trisomy 21 (Tanzi et al., Science 235:880 (1987); Hyman et al., Proc. Natl. Acad. Sci. USA 92:3586-3590 (1995)). Multiple APP isoforms can be generated by alternatively splicing of mRNAs. The major isoforms in brain are APP695, APP751, and APP770 containing 695, 751 and 770 amino acids, respectively. These isoforms are transmembranous proteins having large extracellular regions, with hydrophobic membrane spanning domains and short cytoplasmic segments.

ABCA1 encodes an ATP-binding cassette protein that promotes transport of cholesterol and phospholipids from intracellular compartments to the extracellular space, where it can complex with HDL, lipid-deficient apoA1, and other apolipoproteins (1). Disruption of ABCA1, by Tangier disease mutations in humans or by engineered knock-out in mice, impairs intracellular transport and secretion of lipids (2-5). Like many genes involved in cholesterol homeostasis, ABCA1 is regulated by the liver X receptors (LXR) (6-8), nuclear receptors activated by oxysterols. ABCA1 is also regulated by PPARδ, which can be activated by fatty acid metabolites (9). Both of these classes of receptors form heterodimers with retinoid X receptors (RXR), which bind retinoic acid; as heterodimers, they alter gene transcription. These systems for ABCA1 induction help decrease cellular cholesterol after cholesterol loading (10).

The importance of cerebral cholesterol metabolism in Alzheimer's disease (AD) risk and pathogenesis is supported by genetic, cell culture, mouse model, and epidemiologic data. Apolipoprotein E (apoE) in the CNS is implicated in supplying appropriate membrane lipid for development, nerve growth, and responses to injury and repair in the CNS (11); allelic polymorphisms in the APOE gene are associated with AD risk (12) and the APOE ε4 allele is associated with increased Aβ deposition in AD brain (13). Aβ formation in cell culture systems is inhibited by cholesterol depletion, HMG-CoA reductase inhibition, and ACAT inhibition (14-16). Amyloid deposition in transgenic mouse models of AD can be accelerated by oral cholesterol loading (17), and reduced by HMG-CoA reductase inhibitors (18). Finally, hypercholesterolemia has been associated with AD risk (19,20), and three recent epidemiological studies suggest that cholesterol-lowering drugs can reduce the risk of developing AD (21-23).

There is great need for better understanding of cholesterol transport and processing in the CNS, particularly because a link between cholesterol and Aβ deposition has been established. Because Aβ is believed to be the causative agent of Alzheimer's Disease, there is a need for compositions and methods which reduce the production of Aβ.

### SUMMARY OF THE INVENTION

We found that the ABCA1 ATP-binding cassette protein is expressed in neurons and glia in vivo and in vitro. We further found that ABCA1 is induced unexpectedly by ligands of LXR, and this induction leads to increased levels of secreted Aβ. These data suggest that increased cholesterol can alter Aβ levels through its effects on ABCA1.

According to one aspect of the invention, methods for reducing the level of Aβ secreted from a brain cell are provided. According to another aspect of the invention, methods for modulating cholesterol efflux in a brain cell are provided. The methods include contacting a mammalian brain cell with an agent that reduces expression or activity of a liver X receptor (LXR) protein and/or an ABCA1 ATP-binding cassette protein. In some embodiments, the agent is an agent that reduces LXR and/or ABCA1 protein activity, preferably an agent binds to the LXR and/or ABCA1 protein, and more preferably an antibody or an antibody fragment containing an antigen binding domain that binds to LXR and/or ABCA1 protein. In other embodiments, the agent is an antagonist of LXR and/or ABCA1 function, such as geranylgeranyl pyrophosphate (GGPP). In still other embodiments, the agent is an agent that reduces LXR and/or ABCA1 protein expression. Such agents preferably are molecules that induce RNA inhibition (RNAi) of LXR and/or ABCA1, antisense oligonucleotides, PPARδ modulators, or agents that reduce oxysterol and/or retinoic acid levels in the brain cell. In certain embodiments, the latter agents are statin compounds, or inhibitors of a cytochrome P450 enzyme that generates oxysterols; preferably the cytochrome P450 enzyme is CYP46 that makes 24-hydroxycholesterol. The contacting can occur in vitro or in vivo. Preferred brain cells includes neuron and glial cells.

According to still another aspect of the invention, methods for reducing the rate of onset or the severity of Alzheimer's disease in a subject are provided. The methods include administering to the subject an effective amount of one or more agents selected from the group consisting of: agents that decrease LXR expression or activity; and agents that decrease ABCA1 expression or activity, as described above. In certain embodiments, the subject is a human. In other embodiments, the methods also include administering to the subject an effective amount of a therapeutic agent for treating Alzheimer's disease selected from the group consisting of acetylcholine esterase inhibitors, beta- and gamma-secretase inhibitors, Abeta vaccines, Cu—Zn chelators, cholesterol-lowering drugs and non-steroidal anti-inflammatory drugs.

Compositions are provided in a further aspect of the invention. The compositions are useful for reducing Aβ secretion from a brain cell and include one or more agents that reduce LXR activity or expression and/or one or more agents that reduce ABCA1 activity or expression, as described above. In certain embodiments, the compositions also include a pharmaceutically acceptable carrier. In other embodiments, the compositions also include a therapeutic agent for treating Alzheimer's disease, preferably one selected from the group consisting of acetylcholine esterase inhibitors, beta- and gamma-secretase inhibitors, Abeta vaccines, Cu—Zn chelators, cholesterol-lowering drugs and non-steroidal anti-inflammatory drugs.

In a further aspect of the invention, kits are provided that include any of the foregoing compositions along with instructions for administering the composition to a subject having or suspected of having Alzheimer's disease.

Use of the foregoing compounds and agents in the preparation of medicaments is also provided, particularly for use in treatment of Alzheimer's disease.

Other aspects, embodiments features and advantages of the present invention will be set forth in the detailed description of preferred embodiments that follows, and in part will be apparent from the description or may be learned by practice of the invention.

### BRIEF DESCRIPTION OF THE DRAWINGS

FIG. 1 shows ABCA1 in situ hybridization in rat brain. Sections of rat brain were probed with a radiolabeled ABCA1 antisense probe. FIG. 1A. Coronal section. FIG. 1B. Liver (positive control). FIG. 1C. Sagittal section. FIG. 1D. Sagittal section, sense probe (negative control).

FIG. 2 depicts up-regulation of ABCA1 mRNA in mouse brain after AMPA lesion. In situ hybridization for ABCA1 was conducted on coronal sections of mouse brain after a unilateral hippocampal AMPA lesion. FIG. 2A. Unlesioned brain. FIG. 2B. One hour post-lesion. FIG. 2C. Seven days post-lesion. FIG. 2D. Eleven days post-lesion.

FIG. 3 shows regulation of ABCA1 expression in neuronal cells. Mouse primary neurons (FIG. 3A) and Neuro2A cells (FIGS. 3B, C) were treated in culture with retinoic acid (RA) and oxysterols, which induce gene transcription through RXR and LXR nuclear hormone receptors. Expression of ABCA1 in cell extracts was analyzed by immunoblot analysis. As a control, Neuro2A cells transiently overexpressing ABCA1 from an expression vector demonstrated strong immunoreactivity of approximately 220 kDa (right lanes). FIG. 3A. Primary neurons were treated with 10⁻⁷ or 10⁻⁵ M (0.1 or 10 μM) RA and 22-hydroxycholesterol (22). FIG. 3B. Neuro2A cells treated with (+) or without (−) 10 μM RA and 22-hydroxycholesterol. FIG. 3C. Neuro2A cells treated with 5 μM RA and 5 μM oxysterols (untreated control (C); 22-hydroxycholesterol (22); 25-hydroxycholesterol (25); 7-ketocholesterol (7)). Location of 250 kDa molecular weight marker is noted at left.

FIG. 4 depicts regulation of ABCA1 expression in glial cells. Mouse Neuro2A, mouse BV-2, and rat C6 cells were treated with vehicle (control, lanes 1 and 2), 5 μM retinoic acid and 5 μM 22-hydroxycholesterol (lanes 3 and 4), 1 μM T0-901317 (lanes 5 and 6), or 5 μM retinoic acid and 1 μM TO-901317 (lanes 7 and 8). Cells extracts were collected and analyzed for ABCA1 protein by immunoblot. The molecular weight marker denotes 190 kDa.

FIG. 5 shows ABCA1 distribution in Neuro2A cells. Mouse Neuro2A cells were transiently transfected with an ABCA1-GFP expression construct. Green fluorescence was visualized one day after transfection.

FIG. 6 depicts induction of ABCA1 and secreted Aβ levels. Neuro2A cells were treated with retinoic acid and either 22-hydroxycholesterol (22) or TO-901317 (TO) for one day. Conditioned media was analyzed for Aβ40 and Aβ42 and compared to levels found in cells treated only with vehicle (defined as 100%). Cells treated with 22-hydroxycholesterol showed increased Aβ40 (29%, p<0.02) and Aβ42 (65%, p<0.005). Cells treated with TO-901317 showed increased Aβ40 (25%, p<0.1, NS) and Aβ42 (126%, p<0.001).

FIG. 7 shows RNA inhibition of ABCA1 and secreted Aβ levels. Neuro2A cells were treated with vehicle (ethanol) or retinoic acid and 22-hydroxycholesterol in the presence or absence of RNAi. Control cells showed significant increases in Aβ40 (29%, p<0.05) and Aβ42 (62%, p<0.01) with retinoic acid and 22-hydroxycholesterol. These increases were diminished and non-significant in the presence of RNAi directed against ABCA1: Aβ40, 4%, p<0.9, NS; Aβ42, 37%, p<0.2, NS. RNAi directed against human hsp70 did not diminish the soluble Aβ increases induced by retinoic acid and 22-hydroxycholesterol: Aβ40, 30%, p<0.2, NS; Aβ42, 88%, p<0.02.

### DETAILED DESCRIPTION OF THE INVENTION

The present invention describes the effect of agonists of liver X receptor (LXR) on the expression of the ATP-binding cassette transporter ABCA1 and on the secretion of soluble Aβ in brain. It follows that interfering with LXR expression or activity (particularly transcriptional activity) and/or ABCA1 expression or activity (particularly its transport activity) alters Aβ secretion, and therefore is a therapeutic mechanism for Alzheimer's disease.

Agents that inhibit LXR and/or ABCA1 expression and/or activity thus are useful in accordance with the invention for reduction of Aβ secretion and treatment of Alzheimer's disease.

As used herein, an agent is said to reduce or inhibit the expression or activity of LXR if expression or activity of LXR is less in the presence of the agent than when the agent is absent. Similarly, an agent is said to reduce or inhibit the expression or activity of ABCA1 if expression or activity of ABCA1 is less in the presence of the agent than when the agent is absent. Suitable agents exhibiting these properties include, but are not limited to, peptides, antibodies, carbohydrates, nucleic acids, pharmaceutical agents, and the like, including derivatives thereof.

The agents of the present invention may be identified and/or prepared according to any of the methods and techniques known to those skilled in the art. These agents, particularly peptide agents and antibody agents, may occur or be produced as monomer, dimers, trimers, tetramers or multimers. Such multimers can be prepared using enzymatic or chemical treatment of the native receptor molecules or be prepared using recombinant techniques. Preferably, the agents of the present invention are selected and screened at random or rationally selected or designed using protein modeling techniques.

For random screening, candidate agents are selected at random and assayed for their ability to reduce the expression or activity of LXR and/or ABCA1. Any of the suitable methods and techniques known to those skilled in the art may be employed to assay candidate agents.

For rational selection or design, the agent preferably is selected based on the structure of the LXR or ABCA1 proteins, or on the structure of existing agonists or antagonists of these proteins. Nucleic acid antagonists, such as antisense oligonucleotides or molecules that induce RNA inhibition (RNAi) can be designed based on the sequences of the nucleic acids encoding the LXR or ABCA1 proteins. Any of the suitable methods and techniques known to those skilled in the art may be employed for rational selection or design. For example, one skilled in the art can readily adapt currently available procedures to generate antibodies, peptides, pharmaceutical agents and the like capable of binding to LXR or ABCA1 proteins. Illustrative examples of such available procedures are described, for example, in Hurby et al., “Application of Synthetic Peptides: Antisense Peptides,” in Synthetic Peptides, A User's Guide, W. H. Freeman, N.Y., pp. 289-307 (1992); Kaspczak et al., Biochemistry 28:9230 (1989); and Harlow, Antibodies, Cold Spring Harbor Press, N.Y. (1990).

Illustrative examples of agents of the present invention that inhibit LXR expression or function include, but are not limited to: antibodies that specifically bind to LXR protein or antigen-binding fragments or derivatives thereof; small molecule modulators of LXR function including geranylgeranyl pyrophosphate (GGPP) and the compounds disclosed in U.S. Pat. No. 6,316,503; double stranded LXR RNA molecules for RNA inhibition (RNAi); antisense oligonucleotides that bind to LXR nucleic acid molecules; and agents that reduce oxysterol and/or retinoic acid levels in a brain cell, including statin compounds (e.g., simvastatin), inhibitors of a cytochrome P450 enzyme that generates oxysterols, and PPARδ modulators (e.g., as disclosed in U.S. Pat. No. 6,436,993). The P450 inhibitor compounds may include compounds that are known to inhibit P450 enzymes, such as antidepressants (e.g., fluoxetine, paroxetine, and quinidine against CYP2D6, fluvoxamine against CYPIA2 and CYP2C19, nefazodone against CYP3A4); calcium channel blockers (e.g., mibefradil against CYP1A2, 2D6, and 3A4); and anti-ulcer medications (e.g., cimetidine), as well as compounds of similar structure. Preferably the P450 inhibitor compound inhibits the cytochrome P450 enzyme CYP46, which makes 24-hydroxycholesterol.

Similarly, agents of the present invention that inhibit ABCA1 ATP-binding cassette protein expression or function include, but are not limited to: antibodies that specifically bind to ABCA1 protein or antigen-binding fragments or derivatives thereof; small molecule modulators of ABCA1 function; and molecules that inhibit expression of ABCA1, including double stranded ABCA1 RNA molecules for RNA inhibition (RNAi); antisense oligonucleotides that bind to ABCA1 nucleic acid molecules; and agents that reduce oxysterol and/or retinoic acid levels in a brain cell as described above, including statin compounds, inhibitors of a cytochrome P450 enzyme that generates oxysterols, and PPARδ modulators.

In certain embodiments of the invention, the methods can further include administration of an effective amount of additional therapeutic agent(s) for treating Alzheimer's disease. Such Alzheimer's disease therapeutics include: acetylcholine esterase inhibitors (including donepezil, rivastigmine and galantamine), beta- and gamma-secretase inhibitors, Abeta vaccines, Cu—Zn chelators, cholesterol-lowering drugs and non-steroidal anti-inflammatory drugs.

The antibodies of the present invention include polyclonal and monoclonal antibodies, as well as antibody fragments and derivatives that contain the relevant antigen binding domain of the antibodies. Such antibodies or antibody fragments are preferably used in the diagnostic and therapeutic embodiments of the present invention.

Suitable monoclonal and polyclonal antibodies may be prepared by any of the methods and techniques well known in the art, such as described in, for example, A. M. Campbell, Monoclonal Antibody Technology: Laboratory Techniques in Biochemistry and Molecular Biology, Elsevier Science Publishers, Amsterdam, The Netherlands (1984) and Harlow, Antibodies, Cold Spring Harbor Press, N.Y. (1989). For example, an antibody capable of binding to a domain of LXR or ABCA1 can be generated by immunizing an animal with a polypeptide containing that domain. Any animal (mouse, rabbit, etc.) which is known to produce antibodies can be utilized to produce antibodies with the desired specificity and suitable methods for immunization of these animals are well known in the art, including, for example, subcutaneous or intraperitoneal injection of the polypeptide. One skilled in the art will recognize that the amount of polypeptide used for immunization will vary based on a number of factors, including the animal which is immunized, the antigenicity of the polypeptide selected, and the site of injection.

The polypeptides used as an immunogen may be modified as appropriate or administered in an adjuvant in order to increase the peptide antigenicity. Suitable methods increasing antigenicity are well known in the art, and include, for example, coupling the antigen with a heterologous protein (such as globulin or β-galactosidase) or through the inclusion of an adjuvant during immunization.

A preferred method of generating monoclonal antibodies comprises removing spleen cells from the immunized animals, fusing these cells with myeloma cells, such as SP2/0-Ag14 myeloma cells, and allowing them to become monoclonal antibody-producing hybridoma cells. Any one of a number of methods well known in the art may be used to identify the hybridoma cell which produces an antibody with the desired characteristics. These include screening the hybridomas with an ELISA assay, western blot analysis, or radioimmunoassay (Lutz et al., Exp. Cell Res. 175:109-124 (1988); Kishimoto et al., Proc. Natl. Acad. Sci USA 87:2244-2248 (1990)). Hybridomas secreting the desired antibodies are cloned and the class and subclass of the secreted antibodies are determined using procedures known in the art (Campbell, A. M., Monoclonal Antibody Technology: Laboratory Techniques in Biochemistry and Molecular Biology, Elsevier Science Publishers, Amsterdam, The Netherlands (1984)).

For polyclonal antibodies, antibody-containing antisera is preferably isolated from the immunized animal and is screened for the presence of antibodies with the desired specificity using one of the above-described procedures.

The present invention further provides hybrid cell lines which secrete monoclonal antibodies selective for the LXR protein and/or ABCA1 protein. These monoclonal antibodies are capable of wholly or partially neutralizing the activity of the polypeptides or specifically binding to one of the said polypeptides. These monoclonal antibodies can be used for qualitative and/or quantitative measurement or for purification of the polypeptides according to the invention. The present invention therefore also includes test systems which contain the monoclonal antibodies herein described.

Antibodies may be used as an isolated whole antibody, or can be used as a source for generating antibody fragments which contain the antigen binding site of the antibody. Examples of such antibody fragments include, but are not limited to the Fᵥ, the F(ab), the F(ab)₂, fragment, as well as single chain antibodies.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

Various methods known in the art can be used to generate such fragments without undue experimentation. Recombinant techniques are preferred for generating large quantities of antibodies, antibody fragments and single chain antibodies, as described, for example, in Pluckthum, Bio/Technology 10:163-167 (1992); Carter et al., Bio/Technology 10:167-170 (1992); and Mullinax et al., Biotechniques 12:864-869 (1992). In addition, recombinant techniques may be used to generate heterobifunctional antibodies.

In general, recombinant production of antibodies, antibody fragments or derivatives thereof, uses mRNA encoding an antibody which is isolated from hybridoma cells that produce the desired antibody. This mRNA is then used as a source for generating a cDNA molecule which encodes the antibody, or a fragment thereof. Once obtained, the cDNA may be amplified and expressed according to known methods in a variety of eukaryotic and prokaryotic hosts.

The present invention further includes derivatives of antibodies (antibody derivatives). As used herein, an “antibody derivatives” contain an antibody of the present invention, or a fragment thereof, as well as an additional moiety which is not normally a part of the antibody. Such moieties may improve the solubility, absorption, biological half-life, etc., of the antibody, decrease the toxicity of the antibody, eliminate or attenuate any undesirable side effect of the antibody, or serve as a detectable marker of the presence of the antibody. Moieties capable of mediating such effects are well known in the art.

Detectably labeled antibodies constitute a special class of the antibody derivatives of the present invention. An antibody is said to be “detectably labeled” if the antibody, or fragment thereof, is attached to a molecule which is capable of identification, visualization, or localization using known methods. Suitable detectable labels include radioisotopic labels, enzyme labels, non-radioactive isotopic labels, fluorescent labels, toxin labels, affinity labels, chemiluminescent labels and nuclear magnetic resonance contrast agents.

Illustrative examples of suitable enzyme labels include, but are not limited to, luciferase, malate dehydrogenase, staphylococcal nuclease, delta-5-steroid isomerase, yeast-alcohol dehydrogenase, alpha-glycerol phosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include, but are not limited to, ³H, ¹¹¹In, ¹²⁵I, ¹³¹ ³²P, ³⁵S, ¹⁴C, ⁵¹Cr, ⁵⁷To, ⁵⁸Co, ⁵⁹Fe 75Se ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radionucleotide has a more favorable gamma emission energy for imaging (Perkins et al., Eur. J. Nucl. Med 10:296-301 (1985); Carasquillo et al., J. Nucl. Med. 28:281-287 (1987)). For example, ¹¹¹In coupled to monoclonal antibodies with 1-(p-isothiocyanatobenzyl)-DPTA has shown little uptake in non-tumorous tissues, particularly the liver, and therefore enhances specificity of tumor localization (Esteban et al., J. Nucl. Med. 28:861-870 (1987)).

Illustrative examples of suitable non-radioactive isotopic labels include, but are not limited to, ¹⁵⁷Gd, ⁵⁵Mn, ¹⁶²Dy, ⁵²Tr, and ⁵⁶Fe. Illustrative examples of suitable fluorescent labels include, but are not limited to, an ¹⁵²Eu label, a fluorescent protein (including green fluorescent protein (GFP), enhanced green fluorescent protein (EGFP) and yellow fluorescent protein (YFP)), a fluorescein label, an isothiocyanate label, a rhodamine label, a phycoerytlirin label, a phycocyanin label, an allophycocyanin label, an o-phthaldehyde label, and a fluorescamine label.

Suitable chemical toxins or chemotherapeutic agents include members of the enediyne family of molecules, such as calicheamicin and esperamicin. Chemical toxins can also be taken from the group consisting of duocarmycin (see e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Toxins that are less preferred in the compositions and methods of the invention include poisonous lectins, plant toxins such as ricin, abrin, modeccin, botulinum and diphtheria toxins. Of course, combinations of the various toxins could also be coupled to one antibody molecule thereby accommodating variable cytotoxicity. Other chemotherapeutic agents are known to those skilled in the art.

Illustrative examples of chemiluminescent labels include a luminal label, an isoluminal label, an aromatic acridinium ester label, an imidazole label, an acridiniulll salt label, an oxalate ester label, a luciferin label, a luciferase label, and an aequorin label.

Illustrative examples of nuclear magnetic resonance contrasting agents include paramagnetic heavy metal nuclei such as Gd, Mn, and Fe.

The coupling of one or more molecules to antibodies is envisioned to include many chemical mechanisms, for instance covalent binding, affinity binding, intercalation, coordinate binding, and complexation.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In preferred embodiments, it is contemplated that one may wish to first derivatize the antibody, and then attach the toxin component to the derivatized product. Suitable cross-linking agents for use in this manner include, for example, SPDP (N-succinimidyl-3-(2-pyridyldithio)propionate), and SMPT, 4-succinimidyl-oxycarbonyl-α-methyl-α(2-pyridyldithio)toluene.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N″″″-hexaacetic acid) (Deal et al., J. Med. Chem. 42:2988, 1999), which is an effective chelator of radiometals such as ²²⁵Ac.

In yet other embodiments, the antibodies can be chimeric or humanized antibodies. As used herein, the term “chimeric antibody” refers to an antibody, that combines the murine variable or hypervariable regions with the human constant region or constant and variable framework regions. As used herein, the term “humanized antibody” refers to an antibody that retains only the antigen-binding CDRs from the parent antibody in association with human framework regions (see, Waldmann, 1991, Science 252:1657). Such chimeric or humanized antibodies retaining binding specificity of the murine antibody are expected to have reduced immunogenicity when administered in vivo for diagnostic, prophylactic or therapeutic applications according to the invention.

In certain embodiments, the antibodies are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse have been grafted onto human framework sequences (referred to herein as “humanized antibodies”). Human antibodies directed against PSMA can be generated using transgenic mice carrying parts of the human immune system rather than the mouse system.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

The agents of the present invention may be used in vitro and/or in vivo to study LXR or ABCA1 activity (such as cholesterol efflux in brain cells) and to reduce the rate of onset and/or ameliorate the duration and severity of Alzheimer's disease.

For in vivo use, the agents of the present invention may be provided to a patient as a means of reducing the amount or rate of Aβ secretion.

The present invention therefore provides pharmaceutical compositions comprising an agent that inhibits LXR and/or ABCA1 function and/or expression. These pharmaceutical compositions may be administered orally, rectally, parenterally, intravaginally, intraperitoneally, topically (as by powders, ointments, drops or transdermal patch), bucally, or as an oral or nasal spray. As used herein, “pharmaceutically acceptable carrier” is intended to mean a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion. One of ordinary skill will recognize that the choice of a particular mode of administration can be made empirically based upon considerations such as the particular disease state being treated; the type and degree of the response to be achieved; the specific agent or composition employed; the age, body weight, general health, sex and diet of the patient; the time of administration and rate of excretion of the agent or composition; the duration of the treatment; drugs (such as a chemotherapeutic agent) used in combination or coincidental with the specific composition; and like factors well known in the medical arts.

Pharmaceutical compositions of the present invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Illustrative examples of suitable aqueous and nonaqueous carriers, diluents, solvents or vehicles include, but are not limited to, water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, parabens, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the therapeutic agent or inhibitor, it is desirable to slow the absorption from, for example, subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are preferably mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents as appropriate.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Illustrative examples of embedding compositions which can be used include polymeric substances and waxes.

The inhibitors of LXR and/or ABCA1 expression and/or function also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also contain adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The agent or inhibitor can also be administered in the form of liposomes. As is known to those skilled in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the agent or inhibitor, stabilizers, preservatives, excipients, and the like. Preferred lipids are phospholipids and phosphatidyl cholines (lecithins), both natural and synthetic. Methods to form liposomes are known in the art. See, e.g., Prescott, ed., METHODS IN CELL BIOLOGY, Volume XIV, Academic Press, New York, N.Y. (1976), p. 33 et seq.

The agents of the present invention can be formulated according to known methods to prepare pharmaceutically acceptable compositions, whereby these materials, or their functional derivatives, are combined in a mixture with a pharmaceutically acceptable carrier vehicle. Suitable vehicles and their formulation, inclusive of other human proteins, e.g., human serum albumin, are well known in the art. In order to form a pharmaceutically acceptable composition suitable for effective administration, such compositions will contain an effective amount of one or more agents of the present invention.

Additional pharmaceutical methods may be employed to control the duration of action. Controlled release preparations may be achieved through the use of polymers to complex or absorb the therapeutic agents of the invention. The controlled delivery may be exercised by selecting appropriate macromolecules (such as polyesters, polyamino acids, polyvinyl, pyrrolidone, ethylenevinylacetate, methylcellulose, carboxymethylcellulose, or protamine sulfate) and methods of incorporation in order to control release. Another possible method to control the duration of action by controlled release preparations is to incorporate antibodies into particles of a polymeric material such as polyesters, polyamino acids, hydrogels, poly(lactic acid) or ethylene vinyl acetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacrylate) microcapsules, respectively, or in colloidal drug delivery systems, for example, liposomes, albumin microspheres, microemulsions, nanoparticles, and nanocapsules or in macroemulsions.

The pharmaceutical formulations of the present invention are prepared, for example, by admixing the active agent with solvents and/or carriers, optionally using emulsifiers and/or dispersants, whilst if water is used as the diluent, organic solvents may be used as solubilizing agents or auxiliary solvents. As described above, the excipients used include, for example, water, pharmaceutically acceptable organic solvents such as paraffins, vegetable oils, mono- or polyfunctional alcohols, carriers such as natural mineral powders, synthetic mineral powders, sugars, emulsifiers and lubricants.

The compositions may be advantageously combined and/or used in combination with hypocholesterolemic therapeutic or prophylactic agents that are different from the LXR and ABCA1 inhibitor compounds. In many instances, administration in conjunction with the subject compositions enhances the efficacy of such agents. Exemplary hypocholesterolemic and/or hypolipidemic agents include: bile acid sequestrants such as quaternary amines (e.g. cholestyramine and colestipol); nicotinic acid and its derivatives; HMG-CoA reductase inhibitors such as mevastatin, pravastatin, and simvastatin; gemfibrozil and other fibrinc acids, such as clofibrate, fenofibrate, benzafibrate and cipofibrate; probucol; raloxifene and its derivatives; the therapeutic agents mentioned elsewhere herein; and mixtures thereof.

One of ordinary skill will appreciate that effective amounts of the inventive therapeutic agents can be determined empirically and may be employed in pure form or, where such forms exist, in pharmaceutically acceptable salt, ester or prodrug form. The agonist or antagonist my be administered in compositions in combination with one or more pharmaceutically acceptable excipients. It will be understood that, when administered to a human patient, the total daily usage of the agents and compositions of the present invention will be decided by the attending physician within the scope of sound medical judgement. The specific therapeutically effective dose level for any particular patient will depend upon a variety of factors including the type and degree of the response to be achieved; the specific agent or composition employed; the age, body weight, general health, sex and diet of the patient; the time of administration, route of administration, and rate of excretion of the agent or composition; the duration of the treatment; drugs (such as a chemotherapeutic agent) used in combination or coincidental with the specific composition; and like factors well known in the medical arts.

Techniques of dosage determination are well known in the art for antibody and peptide agents. In general, it is desirable to provide a patient with a dosage of antibody or peptide agent in the range of from about 1 pg/kg to 10 mg/kg (body weight of patient). The therapeutically effective dose can be lowered if the agent of the present invention is additionally administered with another compound. As used herein, one compound is said to be additionally administered with a second compound when the administration of the two compounds is in such proximity of time that both compounds can be detected at the same time in the patient's serum.

For example, satisfactory results are obtained by oral administration of therapeutic dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging may also be arranged in a patient specific manner to provide a predetermined concentration of an agent in the blood, as determined by the RIA technique. Thus patient dosaging may be adjusted to achieve regular on-going trough blood levels, as measured by RIA, on the order of from 50 to 1000 ng/ml, preferably 150 to 500 ng/ml.

The agents of the present invention are intended to be provided to a patient in an amount sufficient to reduce the amount or rate of expression and/or function of LXR and/or ABCA1, i.e., in an “effective amount.” In certain embodiments, an effective amount will be an amount sufficient to inhibit cholesterol efflux in the brain or brain cells, to inhibit secretion of Aβ in the brain or in brain cells, or to treat Alzheimer's disease to alleviate symptoms, slow the progression of the disease, or to inhibit the pathophysiology of the disease. An amount is said to be sufficient to “reduce the amount or rate of expression and/or function of LXR and/or ABCA1” if the dosage, route of administration, etc. of the agent is sufficient to reduce the amount or rate of expression of LXR and/or ABCA1 nucleic acids and/or to reduce the amount of function of LXR and/or ABCA1 proteins. Such an effect can be assayed, for example, by measuring the amount of cholesterol efflux from brain cells as disclosed in the examples, by measuring the expression of LXR and/or ABCA1 nucleic acids, such as by RT-PCR, by examining the onset of Alzheimer's disease symptoms occurring in vivo, or by correlating in vitro blocking studies with predicted in vivo efficacy.

The administration of the agents of the present invention may be for either prophylactic or therapeutic purpose. When provided prophylactically, the agent is provided in advance of any Alzheimer's disease symptoms. The prophylactic administration of the agent serves to prevent or reduce the rate of onset of symptoms. When provided therapeutically, the agent is provided at (or shortly after) the onset of the appearance of symptoms of actual disease. The therapeutic administration of the agent serves to reduce the severity and duration of Alzheimer's disease.

Additionally, one or more of the agents of the present invention which is used in one of the above-described methods can be detectably labeled prior to use, for example, through the use of radioisotopes, affinity labels (such as biotin, avidin, etc.), enzymatic labels (such as horse radish peroxidase, alkaline phosphatase, etc.) fluorescent labels (such as FITC or rhodamine, etc.), paramagnetic atoms, etc. Procedures for accomplishing such labeling are well-known in the art (L. A. Stemberger et al., J. Histochem. Cytochem. 18:315 (1970); E. A. Bayer et al., Meth. Enzym. 62:308 (1979); E. Engval et al., Immunol. 109:129 (1972); and J. W. Goding, J. Immunol. Meth. 13:215 (1976)).

The materials used in the methods described herein are ideally suited for the preparation of a kit. For example, the present invention provides a compartmentalized kit to receive in close confinement, one or more containers which include an agent capable of inhibiting LXR and/or ABCA1 expression and/or function, and optionally one or more other containers comprising one or more of the following: pharmaceutically acceptably carriers and instructions for combining the carrier and the agent and/or instructions for therapeutic use of the agents.

As used herein, a compartmentalized kit includes any kit in which reagents are contained in separate containers. Illustrative examples of such containers include, but are not limited to, small glass containers, plastic containers or strips of plastic or paper. Particularly preferred types of containers allow the skilled worker to efficiently transfer reagents from one compartment to another compartment such that the samples and reagents are not cross-contaminated and the agents or solutions of each container can be added in a quantitative fashion from one compartment to another. Such containers include, but are not be limited to: a container which contains one or more of the agents of the present invention used in the assay, and containers which contain carrier reagents (such as saline, buffers, etc.).

A further aspect of the present invention concerns DNA molecules which encode for the polypeptide and antibody agents described herein. The starting nucleotide molecules can be obtained by the person skilled in the art using known methods. Moreover, the DNA molecules, where the amino acid sequence is known, may be produced synthetically or by amplification methods such as PCR (Sambrook et al., Molecular Cloning: A Laboratory Manual, Cold Spring Harbor Laboratory Press (1989)). The DNA sequences of the present invention include not only the actual nucleotide sequence used by the organism from which the protein is derived but also includes all degenerate forms which encode a peptide with the desired sequence.

The invention includes DNA sequences which have been modified utilizing methods known in the art, such as those generated by mutation, deletion, transposition or addition.

The present invention further includes DNA vectors which contain the DNA sequences described herein. In particular, these may be vectors in which the DNA molecules described are functionally linked to control sequences which allows expression of the corresponding polypeptides. These are preferably plasmids which can be replicated and/or expressed in prokaryotes such as E. coli and/or in eukaryotic systems such as yeasts or mammalian cell lines. These vectors may also be mammalian viral vectors which can be replicated and/or expressed in eukaryotes such as mammalian cell lines and in the human patient, as “host,” for integration into the cellular genome of the patient and expression as genetic therapy systems.

The invention also includes host organisms transformed with the above vectors. Expression in prokaryotes and eukaryotes may be carried out using techniques known in the art. The DNA sequences according to the invention may be expressed as fusion polypeptides or as intact, native polypeptides. Fusion proteins may advantageously be produced in large quantities. They are generally more stable than the native polypeptide and are easy to purify. The expression of these fusion proteins can be controlled by normal host DNA sequences.

Functional prerequisites of the vector systems comprise, in particular, suitable promoter, termination and polyadenylation signals as well as elements which make it possible to carry out replication and selection in mammalian cell lines. For expression of the DNA molecules according to the invention it is particularly desirable to use vectors which are replicable both in mammalian cells and also in prokaryotes such as E. coli.

The present invention is described in further detail in the following non-limiting example.

### EXAMPLE

Experimental Procedures

Materials. 9-cis-Retinoic acid (R-4643) was purchased from Sigma Chemical Co. (St. Louis, Mo.) and dissolved in ethanol at 10 mM. Oxysterols, 22(R)-hydroxycholesterol (H-9384), 25-hydroxycholesterol (H-1015), and 7-ketocholesterol (C-2394), were purchased from Sigma Chemical Co. and dissolved in ethanol at 10 mM. The LXR agonist, TO-901317, was purchased from Cayman Chemical (Ann Arbor, Mich.) and dissolved in ethanol. Anti-ABCA1 rabbit polyclonal antibody was generated against human ABCA1 residues 2071-2261 fused to a histidine tag (24). A second anti-ABC1 polyclonal antibody against a different ABCA1 epitope (amino acid residues 1201-1211 of the human ABC 1 gene [AETSDGTLPAP; SEQ ID NO: I]; catalog number NB 400-105) was obtained from Novus Biologicals (Littleton, Colo.).

In situ hybridization. Unilateral hippocampal AMPA lesions were performed in 4 mice according to published procedures (25), and were sacrificed at 1, 3, 7, and 11 days post-surgery. Brains were removed by cervical dislocation under ether anesthesia, and sectioned coronally or sagitally at 16 μm on a cryostat onto sterile Probe-On Plus slides (Fisher Scientific, Pittsburgh, Pa.). Two non-lesioned rat brains were also examined. In situ hybridization was performed for ABC1 expression utilizing a probe against Genbank sequence X75926, bases 209-253, according to previously published protocols (26). Sections were fixed for 5 minutes in ice-cold 4% paraformaldehyde and stored in 95% ethanol at 5° C. Sections were hybridized overnight with the [³⁵S]-adenosine (Dupont/NEN, Boston, Mass.) end-labeled 45-mer oligonucleotide probes (10,000 cpm/μ1) at 42° C. in sealed chambers humidified with 50% formamide/0.1% diethylpyrocarbonate water, then washed in 1× standard sodium citrate at 55° C. Slides were exposed to Amersham β-max autoradiography film for 13 days (Amersham Biosciences, Piscataway, N.J.). Sections were dipped and exposed to Amersham LM-1 emulsion for 3 months for cellular resolution.

Cell culture. Mouse Neuro2A (neuroblastoma) cells and human IMR-32 (neuroblastoma) cells (American Type Culture Collection, Manassas, Va.) were cultured in Optimem (Gibco, Invitrogen Life Technologies, Carlsbad, Calif.) supplemented with 10% fetal bovine serum. Mouse BV-2 (microglial) and rat C6 (glioma) cells were cultured in Dulbecco's modified Eagle's medium supplemented with 5% fetal bovine serum. Mouse primary neuronal cultures were generated as described (27). Briefly, cortical neurons were isolated from embryonic day 16 Swiss-Webster mice. Individual cortices was dissociated in calcium-free saline and plated on poly-D-lysine (Sigma) coated tissue culture dishes at the density of 1.5×10⁶ cell/ml. The neurons were grown in Neurobasal Media (Gibco BRL) plus 10% fetal bovine serum. One hour after plating, medium with serum was replaced with medium containing B-27 supplement (Gibco BRL).

Transfections. Full-length ABCA1 cDNA with a green fluorescent protein tag fused to the second amino acid of ABCA1 was expressed from the pcDNA1 expression vector (24). Neuro2A cells were transiently transfected using FuGene 6 (Roche Diagnostics Corporation, Roche Applied Science, Indianapolis, Ind.). Green fluorescence was observed one to two days later using the BioRad Laboratories (Hercules, Calif.) MRC-1024 confocal microscope (excitation at 488 nm, emission at 522 nm).

RNA inhibition. Double stranded RNA (RNAi) was generated homologous to a region of the 5′ end of the coding portion of the mouse ABCA1 gene, 5′-AAG TGG CCT GGC CTC TCT TTA-3′ (SEQ ID NO:2) (Dharmacon Research Inc, Lafayette, Colo.). As a negative control, we used a sequence directed against the human hsp70 gene. Neuro2A cells were transfected with the RNAi with Effectene transfection reagent (Qiagen, Valencia, Calif.) according to manufacturer protocol. After an overnight exposure, RNAi complex was removed, cells were washed one time in growth medium, and the media was replaced with Neurobasal medium with 2% B-27 with or without LXR/RXR agonists for one day. Conditioned media was collected and analyzed for Aβ.

Western blot. Cells were lysed in 250 mM sucrose, 10 mM HEPES (pH 7.4), supplemented with complete protease inhibitors (Sigma Chemical Co). Protein levels were quantified by the BCA assay (Pierce, Rockford, Ill.), and samples of 50 μg were held in SDS loading buffer with 2.5% β-mercaptoethanol for 30 min at room temperature, but were not boiled prior to loading. Proteins were separated by SDS-6% polyacrylamide gel electrophoresis and transferred to nitrocellulose. Blots were probed with the anti-ABC1 antibodies (1:1000), and developed with anti-rabbit linked HRP secondary antibody by chemiluminescence.

Aβanalyses. Cells were cultured in Neurobasal medium with 2% B-27 for one day in the presence or absence of LXR/RXR agonists. Aβ40 and Aβ42 levels in the conditioned medium were determined by ELISA, with BNT77 as a capture antibody and BA23-HRP for Aβ40 or BC05-HRP for Aβ42 as detection antibodies (28). Aβ antibodies for ELISA were the generous gift of Takeda Chemical Industries.

Results

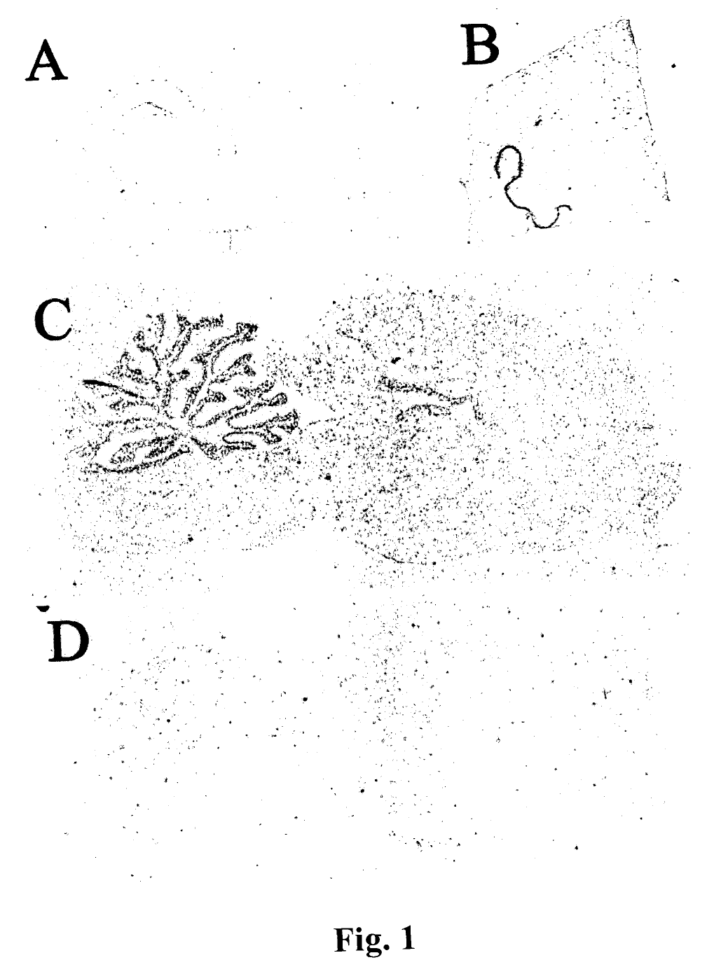

ABCA1 in situ hybridization. We first wanted to determine whether ABCA1 was expressed in brain. In situ hybridization of rat brain demonstrated expression of ABCA1 in rodent brain (FIG. 1). Highest expression was in the neuronal layers of the cerebellum, followed by the hippocampus and cerebral cortex, with low expression in the white matter tracts (FIGS. 1A, C). As a positive control, strong expression was observed in mouse liver (FIG. 1B); as a negative control, the ABCA1 sense probe did not bind to brain sections (FIG. 1D).

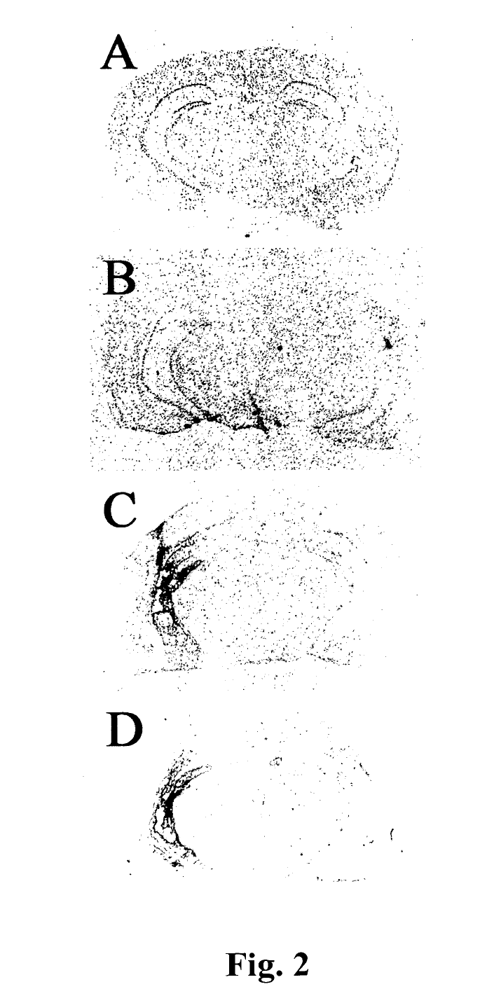

To investigate the regulation of ABCA1 after acute brain damage, we evaluated ABCA1 expression after excitotoxic lesion of the mouse hippocampus (FIG. 2). Stereotactic AMPA lesioning of the hippocampus resulted in an up-regulation of ABCA1 mRNA beginning gradually at 3 days and continuing to increase through 7 and 11 days post-lesion. Emulsion dipped sections demonstrated increased expression in both neuronal and glial elements.

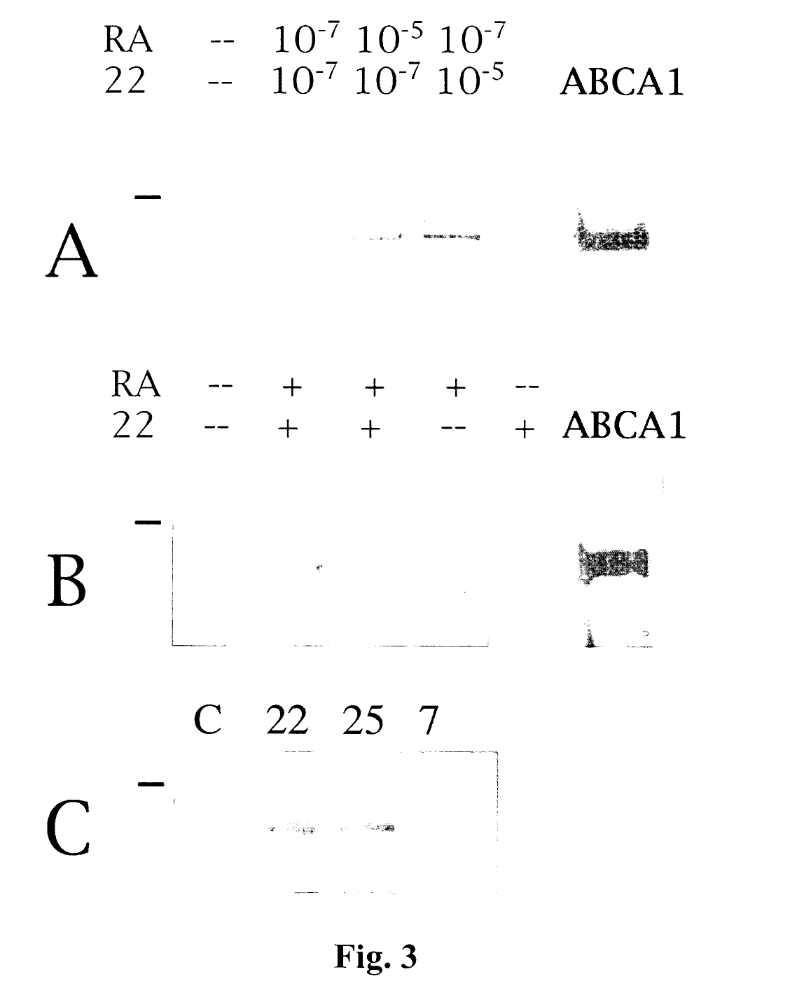

ABCA1 immunoblots. In order to demonstrate ABCA1 protein expression in neurons, we examined proteins isolated from primary cultures of mouse neurons. Western blot analysis using a polyclonal ABCA1 antibody showed a protein of 220 kDa, the size expected from the published sequence (FIG. 3A). This protein co-migrated with ABCA1 from mouse Neuro2A cells transiently transfected with an ABCA1 expression vector (24).

Previous studies demonstrated that ABCA1 was up-regulated by hydroxysterols and retinoic acid (6), which interact with the nuclear receptors, LXR and RXR, respectively. In order to determine whether these agents induce ABCA1 expression in neurons, we treated primary neurons with concentrations of 22-hydroxycholesterol and retinoic acid from 100 nM to 10 uM. One-day treatments with 100 nM of both of these compounds led to an up-regulation of ABCA1 protein compared to untreated cells (FIG. 3A). Increasing concentration of each compound further increased ABCA1 expression (FIG. 3A).

We also examined whether ABCA1 was expressed in an immortalized cell line derived from a mouse neuroblastoma, Neuro2A cells. ABCA1 protein was found at low or undetectable levels in Neuro2A extracts (FIG. 3B). However, when Neuro2A cells were treated with retinoic acid and 22-hydroxycholesterol, expression of ABCA1 was clearly apparent (FIG. 3B). In some experiments, weak up-regulation of ABCA1 was observed in cells treated only with 22-hydroxycholesterol, but never in cells treated only with retinoic acid. Similar experiments with human neuroblastoma IMR-32 cells also showed detectable ABCA1 levels only after induction with retinoic acid and 22-hydroxycholesterol.

We examined whether other oxysterols were capable of increasing ABCA1 expression. Neuro-2A cells were treated with retinoic acid and either 22-hydroxycholesterol, 25-hydroxycholesterol or 7-ketocholesterol for one or two days (FIG. 3C). ABCA1 immunoblots demonstrated that treatment with 25-hydroxycholesterol increased ABCA1 protein nearly as well as 22-hydroxycholesterol, whereas 7-ketocholesterol was not as efficacious. Densitometric analysis demonstrated that 22- and 25-hydroxycholesterol induced ABCA1 to an equivalent extent, but 7-ketocholesterol induced ABCA1 to only half that level.

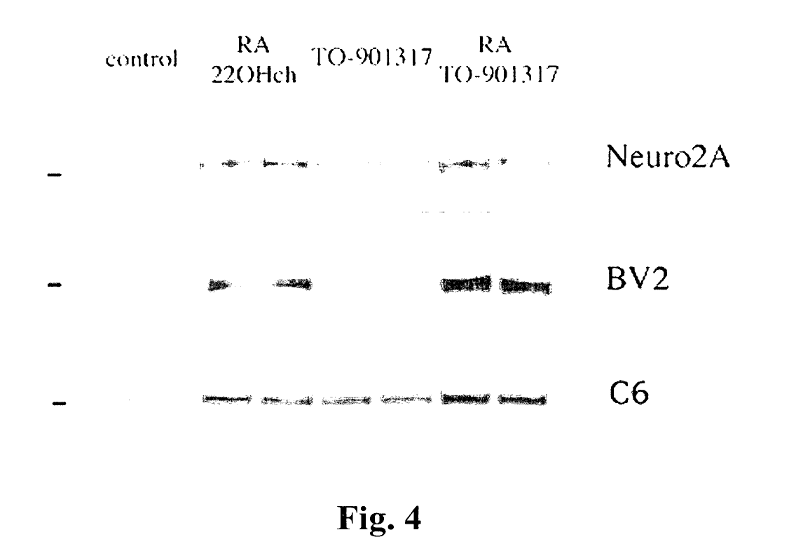

The in situ hybridization data suggested that ABCA1 was also expressed in glial cells after brain lesion. We therefore examined a microglial cell line, BV-2, and a glioma cell line, C-6, for expression of ABCA1 protein. Both glial cell lines expressed detectable levels of ABCA1, and expression was increased after treatment with retinoic acid and 22-hydroxycholesterol (FIG. 4). Expression was also increased after treatment with the non-steroidal LXR agonist TO-901317, and further increased when treated with TO-901317 in the presence of retinoic acid. The response of ABCA1 in glial cells to LXR ligands was similar to that seen in Neuro2A cells (FIG. 4).

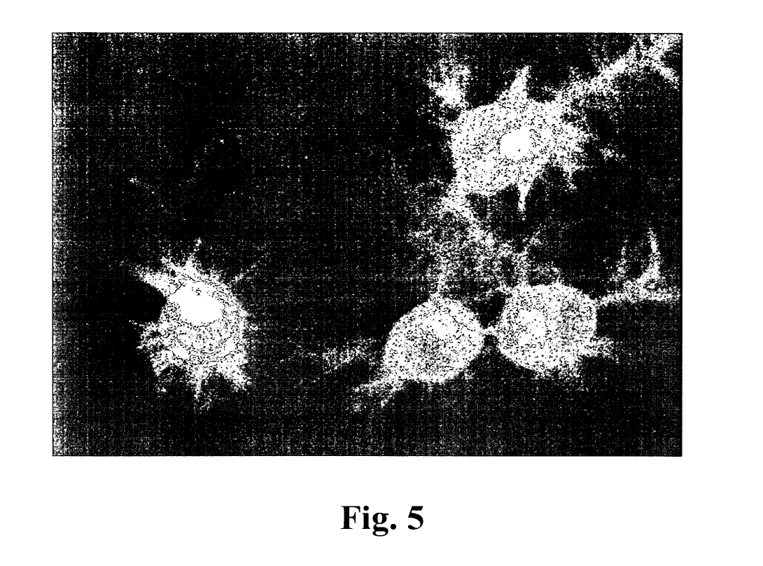

Subcellular localization of ABCA1. In order to visualize the subcellular localization of ABCA1 in Neuro2A cells, we transiently transfected them with an expression vector of full-length ABCA1 fused to green fluorescent protein (24). This membrane-bound protein was found in the endoplasmic reticulum, Golgi apparatus and intracellular vesicles in HeLa cells (29). Similarly, in Neuro2A cells, prominent expression of ABCA1 was observed in the perinuclear compartments and on the cell surface (FIG. 5). ABCA1-GFP was particularly noticeable on processes projecting from the rounded cell bodies (FIG. 5). Thus, ABCA1 is expressed in the plasma membrane of neuronal cells, as expected of a protein whose function is lipid efflux.

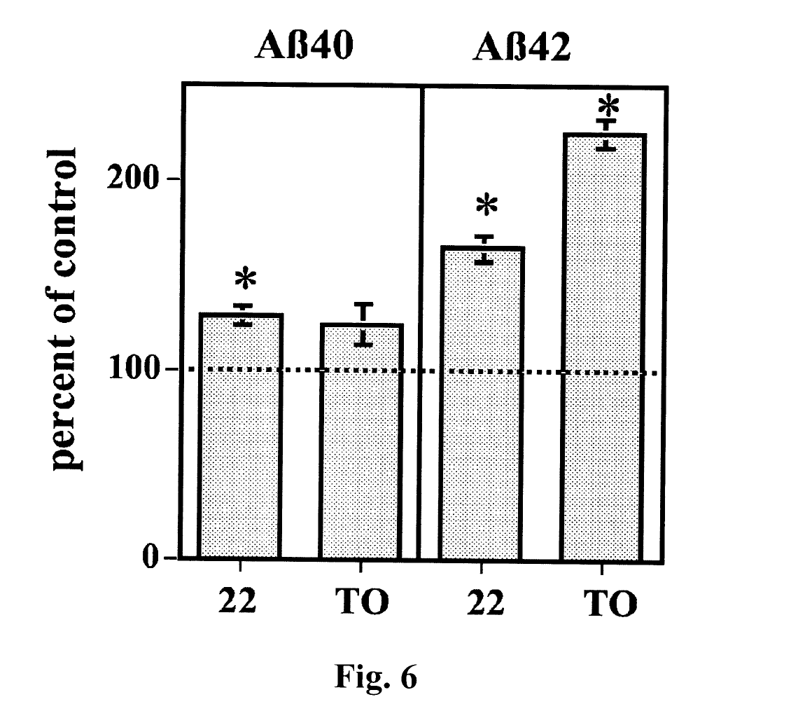

Effects of ABCA1 induction on secreted Aβ levels. Aβ is a 40-42 amino acid, hydrophobic molecule generated by proteolysis of the amyloid precursor protein (APP) (30). We tested whether levels of secreted Aβ were affected by activation of LXR/RXR heterodimers. Neuro2A cells treated with 10 uM 22-hydroxycholesterol and retinoic acid secreted increased levels of both Aβ40 (29%) and Aβ42 (65%) (FIG. 6). Similar increases were observed in experiments with 1 uM or 3 uM 22-hydroxycholesterol. We were concerned that we had altered the cholesterol content of cells in culture with the oxysterol treatments. Therefore, we treated cells with an LXR agonist that was not an oxysterol, TO-901317. Similar increases in secreted Aβ40 (25%) and Aβ42 (126%) were found from cells treated with TO-901317 and retinoic acid (FIG. 6). In all experiments, the increase in Aβ42 was greater than the increase in Aβ40. No differences were observed by western blot analyses in the levels of cellular APP or secreted APP after treatment with LXR and RXR agonists; furthermore, no toxicity was observed by monitoring of released LDH activity.

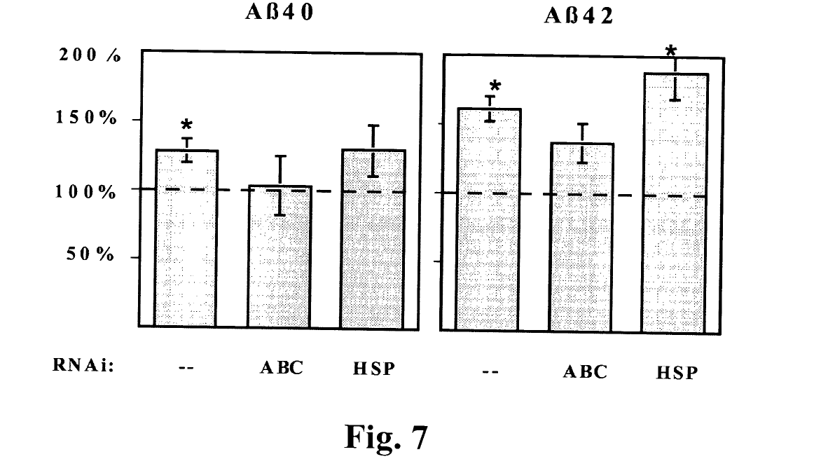

In order to determine whether induction of ABCA1 was partially responsible for the increased levels of secreted Aβ, we treated cells with RNAi to inhibit ABCA1 induction. Western blot analysis showed that RNAi inhibited ABCA1 induction by 30 to 41%. Consistent with the results above, cells treated only with buffer showed significantly increased secretion of Aβ40 and Aβ42 after 22-hydroxycholesterol/retinoic acid treatment. Cells treated with ABCA1 RNAi showed levels of secreted Aβ40 and Aβ42 that were not significantly increased after 22-hydroxycholesterol/retinoic acid treatment (FIG. 7). As a control, we also treated cells with an RNAi against the human hsp70 gene; these cells showed increased levels of secreted Aβ40 and Aβ42 similar to those seen in cells without RNAi. These data suggest that induction of ABCA1 increases levels secreted Aβ.

Discussion

Translating knowledge gained about lipid metabolism in the periphery to the CNS is vital for understanding processes important to neuronal function, degeneration and repair. The cholesterol efflux molecule ABCA1 is expressed widely in the periphery (10,31), but ABCA1 message and protein have also been detected in the brain (10,31-33). Our in situ (FIGS. 1 and 2) and cell culture data (FIGS. 3 and 4) show that ABCA1 expression is high in both neurons and glia. We hypothesized that cholesterol efflux might be very important after neuronal damages, when excess membranes need to be cleared from the brain. Indeed, excitotoxic lesions of the hippocampus resulted in upregulation of ABCA1 mRNA, beginning at 3 days and increasing through 11 days. The time course of increase is similar to apoE and apoJ mRNA, as well as with the development of gliosis (34), suggesting coordinate regulation of these cholesterol transport proteins during repair. Both neurons and glia expressed ABCA1 in response to injury, in contrast to the glial-expressed apoE, suggesting a more ubiquitous role in repair mechanisms.

Oxysterols and agonists of LXR induce ABCA1 expression in many tissue types (6). We found in neuronal cells and glia that ABCA1 was increased by retinoic acid and LXR agonists (FIGS. 3 and 4). Induction of CNS ABCA1 message by LXR agonists was also recently demonstrated in vitro and in vivo (35). 24S-hydroxycholesterol is the most prominent oxysterol in the brain, produced by the brain-specific enzyme cholesterol 24-hydroxylase (CYP46) (36). 24S-hydroxycholesterol induces ABCA1 expression through interactions with either LXRα or LXRβ isoforms (37,38); LXRβ is the isoform more strongly expressed in the brain (35). Better understanding of 24S-hydroxycholesterol regulation will allow better understanding of regulation of brain cholesterol efflux.

Oxysterols, like cholesterol, are components of cell membranes and soluble lipoproteins (39). There are several classes of lipoproteins in the cerebrospinal fluid, defined by their content of apoE and apoAI, and all are high density (40-42). In the CNS, apoE is made by glia (43), and secreted as part of lipoproteins that are smaller and denser than CSF lipoproteins (44). It was suggested that these glial lipoproteins accumulated lipids via cellular cholesterol efflux to become the larger lipoproteins found in the CSF (44). Lipid-poor apolipoproteins, including apoAI and apoE, can interact with ABCA1 to promote cholesterol efflux to generate high-density lipoproteins (45,46). Indeed, cholesterol efflux has been demonstrated from fibroblasts to isolated CSF lipoproteins (47) and from neurons and astrocytes to purified apoE (48). Thus, ABCA1 transfer of lipids to many of the classes of apoE- and apoAI-containing lipoproteins can occur in the CNS, allowing elimination and redistribution of membrane components during degeneration.

Epidemiological studies link high levels of plasma cholesterol with increased risk of Alzheimer's disease (19-23). In vitro and in vivo mouse studies link high levels of cellular cholesterol with high levels of secreted Aβ (14-18). We hypothesized that some of the effects of high cholesterol on Aβ may be due to production of oxysterols from cholesterol, causing changes in gene transcription via interactions with LXR. We focused on changes in ABCA1 because of its significant role in cholesterol efflux and its strong expression in the brain. In neuroblastoma cells, oxysterol induction of ABCA1 was accompanied by increased secreted Aβ species (FIG. 6); a similar effect was seen in C6 glial cells. Importantly, the Aβ produced in these experiments was from endogenous sources of APP, and not from cells overexpressing APP.

The N-terminal 12-14 amino acids of Aβ are hydrophobic, comprising part of the transmembrane domain of APP. Indeed, Aβ cleaved from APP is found in plasma membranes (49). We reasoned that soluble Aβ levels are not only dependent on Aβ production and Aβ degradation, but also on the transfer of Aβ between intracellular and extracellular compartments. Our data that the induction of ABCA1 increased the levels of secreted Aβ (FIGS. 6 and 7) suggests that ABCA1 is a mechanism of Aβ secretion from the cell. We hypothesize that in vivo, Aβ is secreted from the cells associated with cholesterol and phospholipids. Our finding that the more hydrophobic isoform of Aβ, Aβ142, was increased to a greater extent than Aβ40 is consistent with this hypothesis. Increased extracellular Aβ has been observed after brain trauma (50,51); this increase may be due to increased ABCA1 after brain damage (FIG. 2) and increased ABCA1-related efflux of Aβ.

Since LXR agonists induce secreted Aβ species, particularly Aβ42, we hypothesize that an increase in LXR agonists in the brain may increase Aβ deposition. Levels of 24-hydroxycholesterol are increased in CSF of AD patients (52,53), but not in healthy aged individuals (54), suggesting that this compound may increase risk of AD. Furthermore, 24S-hydroxycholesterol is reduced in individuals taking simvastatin (55), who are at decreased risk of AD (21-23). Thus, approaches that reduce brain oxysterol production or act as LXR antagonists may reduce the risk of AD.

In this study, we have characterized the expression and regulation of the cholesterol efflux molecule ABCA1 in neurons and glia. We found that induction of ABCA1 increases secretion of Aβ from cells in culture, suggesting that molecules that regulate ABCA1 could regulate levels of Aβ in the brain. Thus, ABCA1 could constitute a new target in developing therapeutics for prevention of Alzheimer's disease.

### REFERENCES

1. Oram, J. F., and Lawn, R. M. (2001) J Lipid Res 42(8), 1173-9.
2. Walter, M., Gerdes, U., Seedorf, U., and Assmann, G. (1994) Biochem Biophys Res Commun 205(1), 850-6.
3. Rogler, G., Trumbach, B., Klima, B., Lackner, K. J., and Schmitz, G. (1995) Arterioscler Thromb Vasc Biol 15(5), 683-90.
4. Francis, G. A., Knopp, R. H., and Oram, J. F. (1995) J Clin Invest 96(1), 78-87.
5. Orso, E., Broccardo, C., Kaminski, W. E., Bottcher, A., Liebisch, G., Drobnik, W., Gotz, A., Chambenoit, O., Diederich, W., Langmann, T., Spruss, T., Luciani, M. F., Rothe, G., Lackner, K. J., Chimini, G., and Schmitz, G. (2000) Nat Genet 24(2), 192-6.
6. Repa, J. J., Turley, S. D., Lobaccaro, J. A., Medina, J., Li, L., Lustig, K., Shan, B., Heyman, R. A., Dietschy, J. M., and Mangelsdorf, D. J. (2000) Science 289(5484), 1524-9.
7. Costet, P., Luo, Y., Wang, N., and Tall, A. R. (2000) J Biol Chem 275(36), 28240-5.
8. Venkateswaran, A., Laffitte, B. A., Joseph, S. B., Mak, P. A., Wilpitz, D. C., Edwards, P. A., and Tontonoz, P. (2000) Proc Natl Acad Sci USA 97(22), 12097-102.
9. Oliver, W. R., Jr., Shenk, J. L., Snaith, M. R., Russell, C. S., Plunket, K. D., Bodkin, N. L., Lewis, M. C., Winegar, D. A., Sznaidman, M. L., Lambert, M. H., Xu, H. E., Sternbach, D. D., Kliewer, S. A., Hansen, B. C., and Willson, T. M. (2001) Proc Natl Acad Sci US A 98(9), 5306-11.
10. Langmann, T., Klucken, J., Reil, M., Liebisch, G., Luciani, M. F., Chimini, G., Kaminski, W. E., and Schmitz, G. (1999) Biochem Biophys Res Commun 257(1), 29-33.
11. Mahley, R. W. (1988) Science 240, 622-630
12. Strittmatter, W. J., Saunders, A. M., Schmechel, D., Pericak-Vance, M., Enghild, J., Salvesen, G. S., and Roses, A. D. (1993) Proc Natl Acad Sci (USA) 90, 1977-1981
13. Rebeck, G. W., Reiter, J. S., Strickland, D. K., and Hyman, B. T. (1993) Neuron 11, 575-580
14. Simons, M., Keller, P., De Strooper, B., Betreuther, K., Dotti, C. G., and Simons, K. (1998) Proc Natl Acad Sci (USA) 95, 6460-6464
15. Fassbender, K., Simons, M., Bergmann, C., Stroick, M., Lutjohann, D., Keller, P., Runz, H., Kuhli, S., Bertsch, T., van Bergmann, K., Hennerici, M., Beyreuther, K., and Hartmann, T. (2001) Proc Natl Acad Sci (USA) 98, 5856-5861
16. Puglielli, L., Konopka, G., Pack-Chung, E., Ingano, L. A. M., Berezovska, O., Hyrnan, B. T., Chang, T. Y., Tanzi, R. E., and Kovacs, D. M. (2001) Nature Cell Biol 3, 905-912
17. Refolo, L. M., Pappolla, M. A., Malester, B., LaFrancois, J., Thomas-Bryant, T., Wang, R., Tint, G. S., Sambamurti, K., and Duff, K. E. (2000) Neurobiol Dis 7, 321-331
18. Refolo, L. M., Pappolla, M. A., LaFrancois, J., Malester, B., Schmidt, S. D., Thomas-Bryant, T., Tint, G. S., Wang, R., Mercken, M., Petanceska, S. S., and Duff, K. E. (2001) Neurobiol Dis 8, 890-899
19. Notkola, I. L., Sulkava, R., Pekkanen, J., Erkinjuntti, T., Ehnholm, C., Kivinen, P., Tuomilehto, J., and Nissinen, A. (1998) Neuroepidemiology 17, 14-20
20. Kivipelto, M., Helkala, E. L., Laakso, M. P., Hanninen, T., Hallikainen, M., Alhainen, K., Soininen, H., Tuomilehto, J., and Nissinen, A. (2001) Brit Med Journal 322, 1447-1451
21. Jick, H., Zornberg, G. L., Jick, S. S., Seshadri, S., and Drachrnan, D. A. (2000) Lancet 356, 1627-1631
22. Wolozin, B., Kellman, W., Ruosseau, P., Celesia, G. G., and Siegel, G. (2000) Arch Neurol 57, 1439-1443
23. Rockwood, K., Kirkland, S., Hogan, D. B., MacKnight, C., Merry, H., Verreault, R., Wolfson, C., and McDowell, I. (2002) Arch Neurol 59, 223-227
24. Fitzgerald, M. L., Mendez, A. J., Moore, K. J., Andersson, L. P., Panjeton, H. A., and Freeman, M. W. (2001) J Biol Chem 276(18), 15137-45.
25. Page, K., Hollister, R., Tanzi, R. E., and Hyman, B. T. (1996) Proc Natl Acad Sci (USA) 93, 14020-14024
26. Irizarry, M. C., McNamara, M., Fedorchak, K., Hsaio, K., and Hyman, B. T. (1997) J Neuropathol Exp Neurol 56, 965-973
27. Qiu, Z., Strickland, D. K., Hyman, B. T., and Rebeck, G. W. (1999) J Neurochem 73, 1393-1398
28. Fukumoto, H., Tomita, T., Matsunaga, H., Ishibashi, Y., Saido, T. C., and Iwatsubo, T. (1999) NeuroReport 10, 2965-2969
29. Neufeld, E. B., Remaley, A. T., Demosky, S. J., Stonik, J. A., Cooney, A. M., Comly, M., Dwyer, N. K., Zhang, M., Blanchette-Mackie, J., Santamarina-Fojo, S., and Brewer, H. B., Jr. (2001) J Biol Chem 276(29), 27584-90.
30. Hardy, J., and Selkoe, D. J. (2002) Science 297, 353-356
31. Wellington, C. L., Walker, E. K., Suarez, A., Kwok, A., Bissada, N., Singaraja, R., Yang, Y. Z., Zhang, L. H., James, E., Wilson, J. E., Francone, O., McManus, B. M., and Hayden, M. R. (2002) Lab Invest 82(3), 273-83.
32. Luciani, M. F., Denizot, F., Savary, S., Mattei, M. G., and Chimini, G. (1994) Genomics 21(1), 150-9.
33. Lawn, R. M., Wade, D. P., Couse, T. L., and Wilcox, J. N. (2001) Arterioscler Thromb Vasc Biol 21(3), 378-85.
34. Page, K., Hollister, R. D., and Hyman, B. T. (1998) Neurosci 85, 1161-1171
35. Whitney, K. D., Watson, M. A., Collins, J. L., Benson, W. G., Stone, T. M., Numerick, M. J., Tippin, T. K., Wilson, J. G., Winegar, D. A., and Kliewer, S. A. (2002) Mol Enclocrinol 16, 1378-1385
36. Lund, E. G., Guileyardo, J. M., and Russell, D. W. (1999) Proc Natl Acad Sci (USA) 96, 7238-7243
37. Lehmann, J. M., Kliewer, S. A., Moore, L. B., Smith-Oliver, T. A., Oliver, B. B., Su, J.-L., Sundseth, S. S., Winegar, D. A., Blanchard, D. E., Spencer, T. A., and Willson, T. M. (1997) J Biol Chem 272, 3137-3140
38. Janowski, B. A., Grogan, M. J., Jones, S. A., Wisely, G. B., Kliewer, S. A., Corey, E. J., and Mangelsdorf, D. J. (1999) Proc Natl Acad Sci (USA) 96, 266-271
39. Bjorkhem, I., and Diczfalusy, U. (2002) Arterioscler Thromb Vasc Biol 22, 734-742
40. Pitas, R. E., Boyles, J. K., Lee, S. H., Hui, D., and Weisgraber, K. H. (1987) J Biol Chem 262, 14352-14360
41. Borghini, I., Barja, F., Pometta, D., and James, R. W. (1995) Biochim Biophys Acta 1255, 192-200
42. Koch, S., Donarski, N., Goetze, K., Kreckel, M., Stuerenburg, H.-J., Buhmann, C., and Beisiegel, U. (2001) J Lipid Res 42, 1143-1151
43. Pitas, R. E., Boyles, J. K., Lee, S. H., Foss, D., and Mahley, R. W. (1987) Biochim Biophys Acta 917, 148-161
44. LaDu, M. J., Gilligan, S. M., Lukens, J. R., Cabana, V. G., Reardon, C. A., Van Eldik, L. J., and Holtzman, D. M. (1998) J Neurochem 70, 2070-2081
45. Wang, N., Silver, D. L., Costet, P., and Tall, A. R. (2000) J Biol Chem 275(42), 33053-8.
46. Remaley, A. T., Stonik, J. A., Demosky, S. J., Neufeld, E. B., Bocharov, A. V., Vishnyakova, T. G., Eggemman, T. L., Patterson, A. P., Duverger, N. J., Santamarina-Fojo, S., and Brewer, H. B., Jr. (2001) Biochem Biophys Res Commun 280(3), 818-23.
47. Rebeck, G. W., Alonzo, N. C., Berezovska, O., Harr, S. D., Knowles, R. B., Growdon, J. H., Hyman, B. T., and Mendez, A. J. (1998) Exp Neurol 149, 175-182
48. Michikawa, M., Fan, Q.-W., Isobe, I., and Yanagisawa, K. (2000) J Neurochem 74, 1008-1016
49. Mason, R. P., Jacob, R. F., Walter, M. F., Mason, P. A., Avdulov, N. A., Chochina, S. V., Igbavboa, U., and Wood, W. G. (1999) J Biol Chem 274, 18801-18807
50. Roberts, G. W., Gentleman, S. M., Lynch, A., Murray, L., Landon, M., and Graham, D. I. (1994) J Neurol Neurosurg Psychiatry 57, 419-425
51. Uryu, K., Laurer, H., McIntosh, T., Pratico, P., Martinez, D., Leight, S., Lee, V. M.-Y., and Trojanowski, J. Q. (2002) J Neurosci 22, 446-454
52. Schoenknecht, P., Lutjohann, D., Pantel, J., Bardenheuer, H., Hartmann, T., von Bergmann, K., Beyreuther, K., and Schroeder, J. (2002) Neurosci Lett 324, 83-85
53. Papassotiropoulos, A., Lutjohann, D., Bagli, M., Locatelli, S., Jessen, F., Buschfort, R., Ptok, U., Bjorkhem, I., von Bergmann, K., and Heun, R. (2002) J Psych Res 36, 27-32
54. Lutjohann, D., Breuer, O., Ahlborg, G., Nennesmo, I., Siden, A., Diczfalusy, U., and Bjorkhem, I. (1996) Proc Natl Acad Sci (USA) 93, 9799-9804
55. Locatelli, S., Lutjohann, D., Schmidt, H. H. J., Otto, C., Beisiegel, U., and von Bergmann, K. (2002) Arch Neurol 59, 213-216.
   Equivalents

All references disclosed herein are incorporated by reference.

Those skilled in the art will recognize, or be able to ascertain using no more than routine experimentation, many equivalents to the specific embodiments of the invention described herein. Such equivalents are intended to be encompassed by the following claims.

1. A method for reducing the level of Aβ secreted from a brain cell and/or the level of cholesterol efflux from a brain cell, comprising
contacting a mammalian brain cell with an agent that reduces expression or activity of a liver X receptor (LXR) protein and/or an ABCA1 ATP-binding cassette protein.

2. The method of claim 1, wherein the agent is an agent that reduces LXR protein activity and/or ABCA1 protein activity.

3. The method of claim 2, wherein the agent binds to the LXR protein and/or the ABCA1 protein.

4. The method of claim 3, wherein the agent is an antibody or an antibody fragment containing an antigen binding domain that binds to the LXR protein and/or the ABCA1 protein.

5. The method of claim 2, wherein the agent is an antagonist of LXR function and/or ABCA1 function.

6. The method of claim 5, wherein the LXR antagonist is geranylgeranyl pyrophosphate (GGPP).

7. The method of claim 1, wherein the agent is an agent that reduces LXR protein expression and/or ABCA1 protein expression.

8. The method of claim 7, wherein the agent is a molecule that induces RNA inhibition (RNAi).

9. The method of claim 7, wherein the agent is an antisense oligonucleotide.

10. The method of claim 7, wherein the agent reduces oxysterol and/or retinoic acid levels in the brain cell.

11. The method of claim 10, wherein the agent reduces oxysterol levels is a statin compound.

12. The method of claim 11, wherein the agent that reduces oxysterol levels is an inhibitor of a cytochrome P450 enzyme that generates oxysterols.

13. The method of claim 12, wherein the cytochrome P450 enzyme is CYP46 that makes 24-hydroxycholesterol.

14. The method of claim 7, wherein the agent is PPARγ modulator.

15. The method of claim 1, wherein said contacting occurs in vitro.

16. The method of claim 1, wherein the brain cell is a neuron or glial cell.

17-62. (canceled)

63. A method for reducing the rate of onset or the severity of Alzheimer's disease in a subject, comprising
administering to the subject an effective amount of one or more agents selected from the group consisting of: agents that decrease LXR expression or activity; and agents that decrease ABCA1 expression or activity.

64-80. (canceled)

81. The method of claim 63, wherein the brain cell is a neuron or glial cell.

82. The method of claim 63, wherein the subject is a human.

83. (canceled)

84. A composition for reducing Aβ secretion from a brain cell and/or the level of cholesterol efflux from a brain cell, comprising
one or more agents that reduce LXR activity or expression and/or one or more agents that reduce ABCA1 activity or expression.

85-102. (canceled)


SEQUENCE LISTING (2 sequences)

SEQ ID NO: 1 - protein, 11 aa, Homo sapiens
AETSDGTLPAP

SEQ ID NO: 2 - DNA, 21 bp, Mus musculus
aagtggcctggcctctcttta